(19) United States
(12) Patent Application Publication
(10) Pub. No.: US 2024/0141429 A1
(43) Pub. Date: May 2, 2024

(54) METHODS OF MRNA POLY(A) TAIL LENGTH AND HETEROGENEITY ANALYSIS

(71) Applicant: Waters Technologies Corporation, Milford, MA (US)

(72) Inventors: Martin Gilar, Franklin, MA (US); Catalin Doneanu, Franklin, MA (US); Matthew A. Lauber, North Smithfield, RI (US); Mame Maissa Gaye, Cambridge, MA (US)

(73) Assignee: Waters Technologies Corporation, Milford, MA (US)

(21) Appl. No.: 18/498,328

(22) Filed: Oct. 31, 2023

Related U.S. Application Data
(60) Provisional application No. 63/420,827, filed on Oct. 31, 2022.

(51) Int. Cl. C12Q 1/6876; C12Q 1/6806

(52) U.S. Cl. CPC C12Q 1/6876; C12Q 1/6806; C12Q 2600/158; C12Q 2600/166

(57) ABSTRACT

The present disclosure describes methods for analyzing mRNA poly(A) tail sequences with a high resolution to determine poly(A) tail length and heterogeneity. The method involves digesting an mRNA molecule to liberate a poly(A) tail, preparing a chromatographic sample comprising the mRNA poly(A) tails, preparing a second chromatographic sample comprising a reference sequence comprising a mRNA poly(A) tails having a predetermined length, separating the first and second samples by a chromatography method, which result in one or more chromatograms, and determining a sequence length of the mRNA poly(A) tails by comparing the chromatograms of the first and second samples. Chromatography methods for analyzing the poly(A) tail may include ultraviolet size-exclusion chromatography (SEC UV), ultraviolet ion-pair reversed-phase liquid chromatography (IP RP LC UV), ultra high performance liquid chromatography (UPHLC), and combinations thereof.

### RELATED APPLICATIONS

This application claims priority to and benefit of U.S. Provisional Application Ser. No. 63/420,827, filed on Oct. 31, 2023, and entitled “METHODS OF mRNA POLY(A) TAIL LENGTH AND HETEROGENEITY ANALYSIS,” the contents of which are herein incorporated by reference in their entireties.

### FIELD OF THE TECHNOLOGY

The present disclosure relates to methods and techniques for determining poly(A) tail length and heterogeneity.

### BACKGROUND

mRNA may be used for vaccine or protein replacement therapy. The poly(A) tail is often added to the 3′ end of RNA (e.g., mRNA) for controlling in vivo stability.

The poly(A) tail may be added by enzymatic reaction, which often results in heterogeneous poly(A) tail. This means that the resulting poly(A) tail is a mixture of poly(A) molecules that have a varying length of plus or minus about five nucleotides and roughly a gaussian distribution of chain lengths.

One of the goals of mRNA analysis is to characterize the length and heterogeneity of the poly(A) tail. Due to the size and molecular weight of the intact mRNA, it is difficult to perform the analysis with mass spectrometry (MS) or by liquid chromatography (LC) MS techniques.

Therefore, there exists a need to analyze poly(A) tails of mRNA in a simple manner with sufficient resolution to determine length and heterogeneity.

### SUMMARY

mRNA molecules can be cut into smaller and more manageable pieces by enzymatic cleavage. It is possible to cleave the mRNA molecule just prior to the poly(A) tail and liberate the 3′ end poly(A) tail prior to the analysis. The shorter 60-150 poly(A) tail oligonucleotides can be separated from the remaining pieces of mRNA and characterized. Simple chromatographic methods such as LC UV are preferred by the industry.

Alternatively, capillary electrophoresis or other separation methods can be used. LC MS method or MS methods can be also used for confirmation of poly(A) tail length (molecular weight), but such methods require more sophisticated instrumentation and skilled operator than simple LC UV methods and therefore they are less useful in quality control of pharmaceutical compounds.

The present technology uses LC UV analysis as a technique for analyzing the poly(A) tail length and heterogeneity of mRNA. The methods are suitable for routine quality control of mRNA therapeutic compounds. Such methods are highly desirable for the pharma industry and not available today.

The disclosure herein is generally related to methods for analyzing mRNA poly(A) tail sequences with a high resolution to determine poly(A) tail length and heterogeneity.

In an embodiment, the methods determine mRNA poly(A) tail size by digesting an mRNA molecule to liberate a poly(A) tail, preparing a chromatographic sample comprising the mRNA poly(A) tails, preparing a second chromatographic sample comprising a reference sequence comprising a mRNA poly(A) tails having a predetermined length, separating the first and second samples by a chromatography method, which result in one or more chromatograms, and determining a sequence length of the mRNA poly(A) tails by comparing the chromatograms of the first and second samples.

In some embodiments, the mRNA poly(A)tail can be liberated with RNAse T1, RNAse A, or other enzymes that do not cleave A positions. In some embodiments, a combination of enzymes are used to liberate the poly(A) tail. In such cases, a double digest will generate very short oligos that will less likely interfere with SEC measurement of poly(A) tail in case that the poly(A) tail is so short that is approaches oligo sizes generated by one digest only. E.g. RNAse T1 generated in some examples 27 nt long oligo that may interfere with measurement of ˜30 nt poly(A) tail.

In an alternative embodiment, the methods may also be used to determine heterogeneity when studying the chromatograms.

In some embodiments, the reference sequence comprises 30-150 nt in length.

In some embodiments, the methods include selecting a length of the reference sequence by calibrating with a DNA or RNA reference sequence standard.

In some embodiments, the methods discussed herein include preparing the poly(A) tail reference sequence standard by mixing and digesting poly(A) synthetic oligonucleotides and digesting the poly(A) synthetic oligonucleotides to create a full ladder of poly(A) species.

In some embodiments, the methods include preparing the poly(A) tail reference sequence standard with poly(A) polymerases or 3′ or 5′ exonucleases to create a full ladder of poly(A) species.

In some embodiments, the methods further include using the poly(A) tail reference sequence standard to generate a calibration curve and determining the length of a poly(A) clip from the mRNA molecule.

In some embodiments, the mRNA poly(A) tails range between about 80 to about 120 oligonucleotides.

In some embodiments, the mRNA poly(A) tails have a length distribution that is observable from chromatogram with a n/n−1 resolution.

In some embodiments, the methods further include calculating the dispersity of the mRNA poly(A) tails based on one or more poly(A) tail peak widths of the chromatograms.

In some embodiments, the methods are performed with mass spectrometry (MS) compatible mobile phases.

In some embodiments, the digesting step includes liberating the 3′ poly(A) tail of the mRNA molecule by enzymatic or chemical cleavage.

In some embodiments, the methods further include selecting from the one or more mRNA poly(A) tails a sequence length of about 100 nucleotides.

In some embodiments, chromatography methods for analyzing samples include ultraviolet size-exclusion chromatography (SEC UV), ultraviolet ion-pair reversed-phase liquid chromatography (IP RP LC UV), ultra high performance liquid chromatography (UPHLC), and a combination thereof. In some embodiments, one or more of these methods are used with or without UV.

### BRIEF DESCRIPTION OF THE DRAWINGS

The technology will be more fully understood from the following detailed description taken in conjunction with the accompanying drawings, in which:

FIG. 1 shows a diagram of the structural components of a mRNA sequence, including the poly(A) tail portion.

FIG. 2A shows results of IP RP LC MS analysis of RNase T1 digest.

FIG. 2B shows detailed peak results of the chromatogram of FIG. 2A at about 20.5 minutes, demonstrating the appearance of intact mRNA.

FIG. 3 shows SEC pore size optimization results for a 15-150 nt poly DNA standard of synthetic oligonucleotides (crude, not purified).

FIG. 4 shows a graphical example of size exclusion chromatography calibration (SEC) changes from DNA to RNA by adjusting the DNA linear slope and calibration intercept values.

FIG. 5A shows an analysis of SEC 250 Å RNAse T1 digest analysis of EPO mRNA intact. The figure shows assignment of poly(A) tail length from DNA calibration. When RNA calibration is used and verified with MS data, the length comes to 124 nt.

FIG. 5B shows an analysis of SEC 250 Å RNAse T1 digest analysis of Fluc mRNA intact. When used with RNA calibration, the length comes to about 127 nt.

FIG. 6 shows differing results for a mobile phase buffer pH effect based on the acidity of the mobile phases.

FIG. 7 shows differing results for a mobile phase buffer pH effect based on the ammonium acetate concentration.

FIG. 8 shows a comparison of standards based on the type of calibration standard using SEC 250 Å. The figure compares oligo(A) RNA, oligo(A) DNA, and oligo(T) DNA standards.

FIG. 9 shows SEC 250 Å poly(A) analysis for EPO and Flue beta with 100 nt oligo(A) RNA standard is used for calibration.

FIG. 10A shows a LC MS analysis of a poly(A) tail for EPO mRNA T1 RNase digest based on an IP RP LC MS method. These results provide an alternative to SEC.

FIG. 10B shows poly(A) tail LC MS data of FIG. 10A at about 37 minutes, demonstrating a poly(A) tail length of 119-130 nt.

FIG. 11A shows a LC MS analysis of a poly(A) tail for Flue beta mRNA T1 RNase digest based on an IP RP LC MS method for an mRNA digest analysis. These results can indicate the length and polydispersity of poly(A) tail.

FIG. 11B shows poly(A) tail LC MS data of FIG. 10A at about 37 minutes, demonstrating a poly(A) tail length of 120-128 nt.

FIG. 12A shows a LC UV analysis of liberated poly(A) tail using high resolution octylammonium (OAA) acetate IP system. Poly(A) species in the range of ˜100-140 can be resolved and their length assigned using a size standard oligo(A) RNA, e.g. 100 nt and its shorter RNA oligo(A) species.

FIG. 12B shows an example of a calibration curve generated using an RNA oligo(A) calibration standard to calculate poly(A) length N and distribution in the poly(A) tail analysis.

FIG. 13A shows a graphical example of SEC calibration curves obtained for RNA oligo(A) (30-120 nt) and DNA oligo(A) (30-150 nt).

FIG. 13B shows a graphical example of the calibrations of FIG. 13A that are practically superimposable when the retention correction of DNA oligo(A) retention times are adjusted by 0.306 minutes.

### DETAILED DESCRIPTION

The present technology relates to mRNA analytical techniques for determining poly(A) tail length and heterogeneity, useful for the translation and protection of the mRNA molecule.

mRNA length is usually about 2000-5000 nucleotides and about 0.6-1.5 MDa in molecular weight. The poly(a) tail is typically about 60-150 nucleotides long. The present technology uses analytical methods for mRNA quality control and characterization.

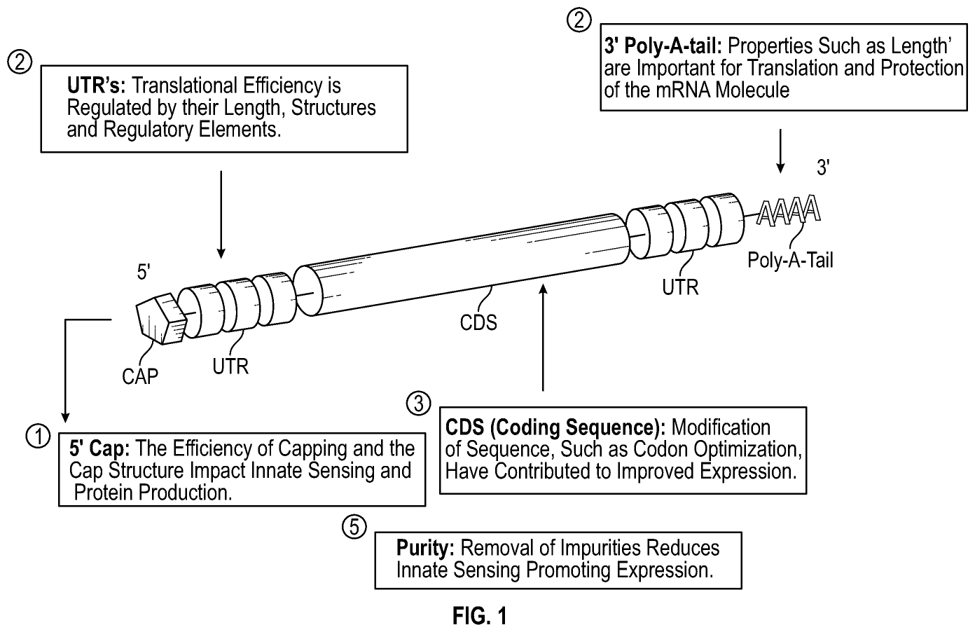

As shown in FIG. 1, the mRNA structure has untranslated regions (UTR on both ends), a coding region in the middle, 5′ end capping structure, and 3′ end poly(A) tail that is heterogeneous and about 60-150 nucleotides long. All the above features have functional and therapeutic importance.

The challenges involved for using mRNA as a therapeutic modality is the large molecular weight of the nucleotide sequence, which is particularly difficult for liquid chromatography (LC) and mass spectrometry (MS) analysis. In particular, LC resolution is poor for intact mRNA molecules and it is difficult to obtain molecular weight using MS due to multiple charges and poly(A) tail microheterogeneity as the poly(A) tail may contain multiple species that slightly differ in length, e.g., by one nucleotide.

The present technology solves the above issues by analyzing the mRNA digest with simple chromatography methods, such as LC UV to analyze the poly(A) tail.

In an embodiment, the methods determine mRNA poly(A) tail size by digesting an mRNA molecule to liberate a poly(A) tail, preparing a chromatographic sample comprising the mRNA poly(A) tails, preparing a second chromatographic sample comprising a reference sequence comprising a mRNA poly(A) tails having a predetermined length, separating the first and second samples by a chromatography method, which result in one or more chromatograms, and determining a sequence length of the mRNA poly(A) tails by comparing the chromatograms of the first and second samples.

In an alternative embodiment, the methods determine mRNA poly(A) tail size by spiking the poly(A) tail sample with a sizing standard. In such a case, the sample and sizing standards are analyzed in a single analysis without the danger of slight retention shifts between analyses, which are advantageous with chromatography methods such as IP RP LC and SEC. The mRNA sizing standard may also be spiked just preceding the eluting poly(A) sample. For example, when the sample is 120 nt, the 100 and/or 110 RNA oligo(A) standards can be spiked into the sample.

The methods address issues with mRNA size. mRNA is a large nucleic acid, with a molecular weight ranging from 0.3-3 MDa. Therefore, it is difficult to analyze by LC and LC MS techniques.

To solve this issue, the present technology may use enzymatic (or chemical) digests to cut large molecule into small oligonucleotides. This technique still produces complex results and, therefore, the resulting mixture typically requires LC MS analysis after enzymatic digestion.

The pharmaceutical and biotechnology industry advantageously benefits from simple and robust (including non-MS) methods for routine quality control (QC) of therapeutical compounds.

The present technology solves the problem of resolving nucleotides such as poly(A) tail (e.g., about 100 nt oligos) and sgRNA (e.g., about 100 nt long) from its impurities (e.g., 99 or 101 nt in length).

Poly(A) chains are difficult to detect due to multiple MS signals (molecules) present. The large oligonucleotides such as poly(A) samples are not resolved from its truncated products. For example, a 100 nt poly(A) chain is not resolved from 99 nt or less chain. Therefore, it is difficult to accurately assign the length of a poly(A) tail and to specify its heterogeneity without using LC MS or MS techniques such as taught with the present technology.

Digestion methods are not limited and may include the use of any number of known nucleases/exonucleases and gene editing techniques such as CRISPR/Cas9 to create a full poly(A) ladder.

In some embodiments, the mRNA poly(A)tail can be liberated with RNAse T1, RNAse A, or other enzymes that do not cleave A positions.

In some embodiments, a combination of enzymes (e.g., a double digest) are used to liberate the poly(A) tail. The benefit of, e.g., a double digest is that this method will generate very short oligos that will less likely interfere with SEC measurement of a poly(A) tail in an embodiment where the poly(A) tail is so short that approaches oligo sizes generated by one digest only. For example, if digestion using an RNAse T1 enzyme generates a 27 nt long oligo, that may interfere with a measurement of a ˜30 nt poly(A) tail.

In some embodiments, the present technology uses RNAse T1 to cleave RNA sequences at the G position and leave phosphate on 3′ end. The extended reaction and sample storage can alter the nature phosphate into its cyclic form. The different nature of the phosphate alters the retention and mass of the resulting oligonucleotide, which may complicate nucleotide analysis.

In some embodiments, phosphatases may be added to remove 5′ and 3′ phosphate groups. In a preferred embodiment, the present technology removes the phosphate groups from the 3′ end of oligonucleotides using Calf Intestinal alkaline Phosphatase (CIP) enzymes.

In some embodiments, the present technology uses polyadenylases to produce a poly(A) tail at the mRNA 3′ end. For example, a polyadenylase can be used to extend a synthetic oligo A standard. This advantageously saves on cost since standards greater than about 60 nt in length are expensive to prepare.

A poly(A) tail length of the methods may range from 60-150 nucleotides long. Preferably, the poly(A) tail length is 100-120 nucleotides long. More preferably, the tail length is about 100 nucleotides long. While these embodiments are preferred, the specific poly(A) tail length is not particularly limited and may vary based on the desired molecule and therapeutic application through routine experimentation as recognized by those skilled in the art.

In an embodiment, the chromatography methods of the present technology may use ultraviolet size-exclusion chromatography (SEC UV), ultraviolet ion-pair reversed-phase liquid chromatography (IP RP LC UV), and/or ultra high performance liquid chromatography (UPHLC) for separating poly(A) tail samples to determine a particular length and heterogeneity. Non-UV methods may also be used. These methods may use mobile phases that are compatible with the samples for MS detection.

In an embodiment, SEC is used for separation as a chromatographic method. SEC column pore size is not limited and may vary based on the nucleotide length for optimal separation. For example, SEC column pore size may be 125 Å, 200 Å, 250 Å, or 450 Å. The present technology identified SEC columns with optimal pore size and particle size for poly(A) size separation. In a preferred embodiment, 125 Å columns may be useful for short oligonucleotides separation (e.g., 2-20 nt length) whereas 200 Å and 250 Å may be suitable for poly(A) separation for a larger range (e.g., 20-150 nt). In a more preferred embodiment, SEC is used with a pore size of 250 Å.

The present technology may use a calibration equation such as in Example 2 to determine optimal SEC UV conditions for poly(A) sizing. From the elution time (or volume) the user can simply calculate the “N”, which is the observed length of the observed poly(A) RNA clip.

In some embodiments, a calibration standard of about 15-150 nt poly(A) species may be used. In a preferred embodiment, the calibration standard is 40-150 nt. This sample would be analyzed by users and a new calibration equation that will be established after initial analysis. One of ordinary skill in the art recognizes customer instruments may have slightly different configurations (e.g., capillary length, different buffer used for separation, etc.) and, thus, the calibration conditions may be modified accordingly.

In some embodiments, an oligo(A)/poly(A) DNA standard is used as a surrogate for a poly(A) RNA standard with two-point or multi-point conversion correction.

In a more preferred embodiment, oligo(A)/poly(A) RNA standard is used since it corresponds exactly to the expected sample. Oligo(A) RNA standard is used as it behaves differently than oligo(A) DNA. Since the sample of interest is oligo(A) (poly(A) tail), no hairpins or self-complementary secondary structure is present to affect hydrodynamic radius of the sample and the SEC retention.

SEC advantageously gives repeatable retention over extensive time. Therefore, the present technology can generate calibration for oligo(A) RNA standard “in factory” and provide it to users.

The present technology can also provide RNA poly(A) standard to users as part of a kit. In some embodiments, selected RNA poly(A) standards are for a predetermined length (e.g., 120 nt, 60 nt, 40, and/or 30 nt). In a preferred embodiment, the selected RNA poly(A) standard is 40 nt. To generate a linear calibration a minimum of two points are required.

The present technology can provide DNA poly(A) standard for calibration with a mathematical conversion to RNA calibration. The peak width and shape observed in SEC can be mathematically deconvoluted to obtain sample heterogeneity information/polymer polydispersity estimation.

In some embodiments, phosphate and/or ammonium acetate are used as buffers in the mobile phases. Replacement of phosphate with ammonium acetate and reduction of ammonium acetate buffer concentration leads to ionic repulsion of negatively charged poly(A) sample from the pores. However, the present technology may counteract this effect by using lower pH and higher ammonium acetate buffer concentrations.

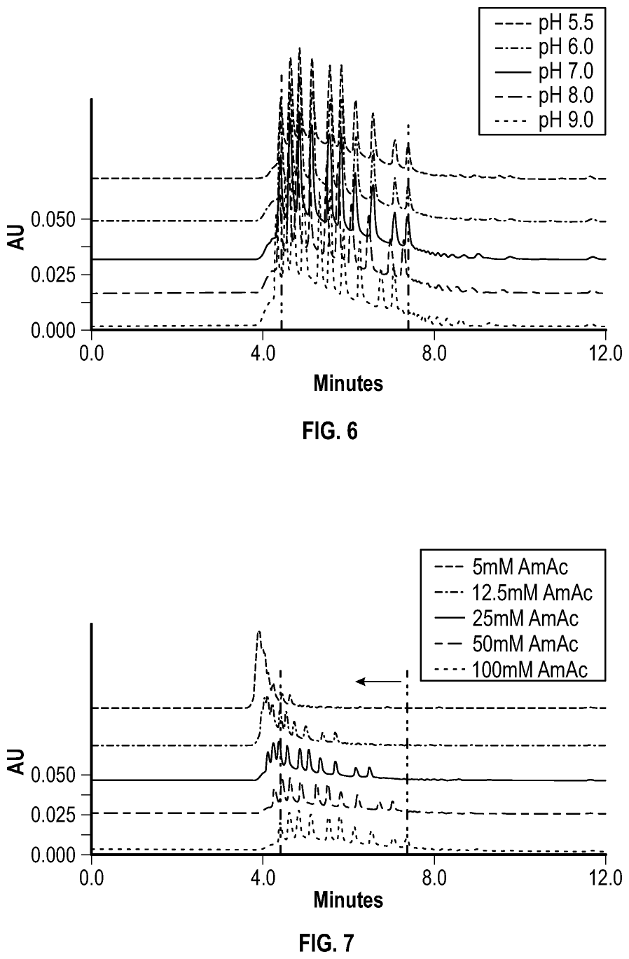

In certain embodiments, the pH of the mobile phase ranges from 4 to 9. In a preferred embodiment, a more acidic pH is preferred. As shown in FIG. 6, different pH mobile phases with SEC 250 Å Premier, 100 mM ammonium acetate were tested. As basic conditions are adjusted to a more acidic pH, mobile phases showed an improvement in results, with 5.5 being optimal. NH₄ Acetate, pH 5.5 mobile phase should reduce silanol charge to a minimum. However, there may be a minor shift compared to phosphate buffer.

In an alternative embodiment, a higher pH buffer may be used with a greater concentration of buffer. Concentrations of ammonium acetate or phosphate buffer are not limited. For example, buffer concentrations may range from 1 mM to 1000 mM. In more preferred embodiments, the buffer concentration is 5, 12.5, 25, 50, or 100 mM. For example, as shown in FIG. 7, with SEC 250 Å Premier, native Ammonium Acetate pH 6.85, ionic repulsion from pores may be obviated based on the ammonium acetate concentration ranging from 5 mM to 100 mM.

Each buffer may require slightly different calibration. For example, because of potential sample adsorption to metal hardware, Premier columns commercially available from Waters Technology Corporation, Milford, MA (i.e., columns with High Performance Surfaces (HPS) to effectively reduce non-specific adsorption (NSA) losses due to metal interactions) are preferred for sample recovery. However, other columns may be utilized with the methods disclosed herein.

In an embodiment, heterogeneity may be observed based on several chromatography method techniques. If an observed poly(A) length is about 100-120 mer, there may yet be a mixture of oligonucleotides that SEC alone would not be sufficient to observe heterogeneity. Sufficient resolution would need to separate about 90-130 nt long species differing in a single nucleotide. For example, at about 124 nt poly(A) RNA tail, there may still be a mixture of oligonucleotides with length centered at about 124 nt. The present technology solves this by further using a IP RP LC technique has high resolution capable of n/n−1 resolution up to about 100-130 mer oligonucleotides. This allows the user to chromatographically observe the heterogeneity of the poly(A) RNA tail even if only using a LC UV detector. This is achieved with high resolution ion-pairing systems, e.g., OAA buffers with shallow (0.1-0.25-0.5% acetonitrile/min) gradients and highly efficient 150×2.1 mm 1.7 um columns.

In an embodiment, RNA may be stabilized to avoid sample degradation. Sample degradation may occur when RNases present in mobile phase are enriched on column during equilibration, T1 RNase present in sample is denatured at a certain temperature (e.g., 60 and may show RNase non-specific activity, and RNA sample is hydrolyzed at high pH mobile phase (pH ˜9) and high temperature (e.g., 60° C.).

In some embodiments, stabilization includes using RNase free water for mobile phase preparation.

In some embodiments, stabilization includes adjusting the mobile phase to a pH of 7.0.

In some embodiments, stabilization includes adding HFIP (protein denaturant), which will reduce enzymatic activity due to HFIP denaturing capabilities and can assist to adjust pH. In a preferred embodiment, 1% HFIP can be added with buffers described herein. HFIP and RNAse free water can be combined to minimize sample degradation.

In some embodiments, the column temperature can be adjusted to thermally denature enzymes. For example, a column temperature of 90° C. or higher may be used at the risk of reduced IP efficiency and resolution of n/n−1 species.

In some embodiments, the poly(A) tail can be resolved into multiple species in a chromatography method (e.g., IP RP LC) based on the peaks. For example, shorter oligonucleotides generated by the digest elute early in IP RP LC. A sample that is heterogeneous (e.g., 124 nt poly(A) with additional 1-10 mer shorter and 1-10 mer longer poly(A) oligonuclotides) may have a “bell-curve” distribution.

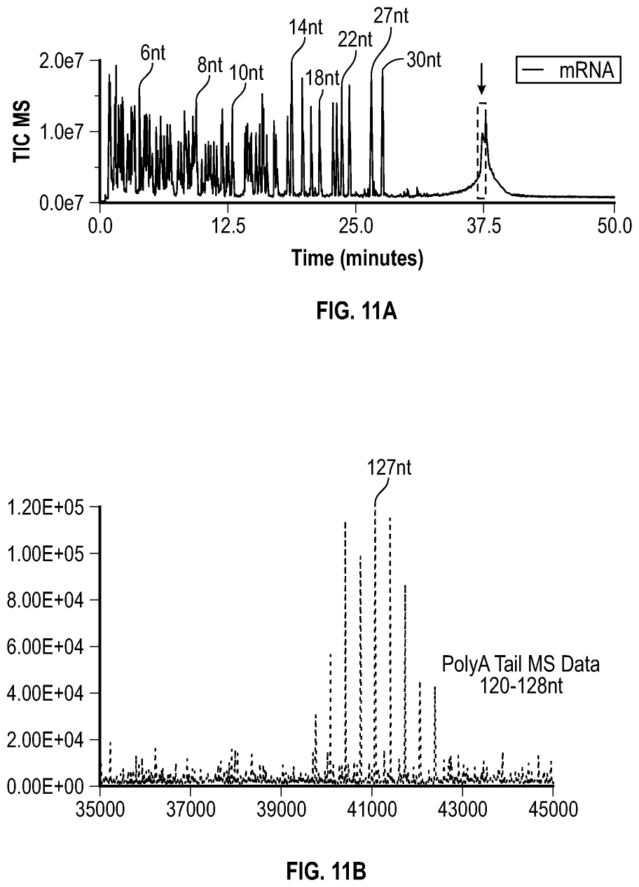

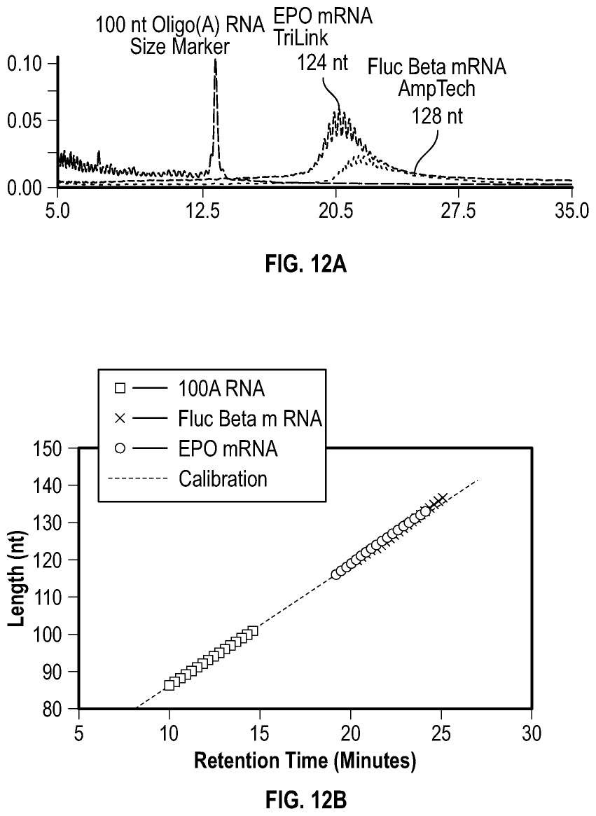

In some embodiments, IP RP LC can be used to monitor a dispersity of poly(A) tail. IP RP LC can be combined with SEC method that provides the average length of poly(A) tail. IP RP LC method with UV detection may use standards to assign the length of observed poly(A) species. For example, as shown in FIG. 12A, the technology uses a 100 nt RNA synthetic oligo(A) with its impurities to create calibration and assign the length and heterogeneity of two different mRNA samples. The accuracy is within 1-2 nt of LC MS data as shown in FIGS. 11 and 12.

In some embodiments, a poly(A) tail length can be determined with a defined RNA standard that is spiked (e.g., 100 mer) to sample. From the observed relative elution based on the length of the poly(A) RNA standard, the length of poly(A) peaks can be calculated. Alternatively, as shown in FIG. 12B, a calibration standard can be used to generate calibration curve and to calculate poly(A) length N and distribution in the poly(A) tail analysis.

In an embodiment, IP RP LC can be used instead of SEC method since it provides both length and distribution of lengths of poly(A) tail. However, SEC method is much simpler and more robust.

In an embodiment, IP RP LC can be optimized to be compatible with MS. In some embodiments, an alkylamine/HFIP buffer system is used to obtain the best resolution and good MS sensitivity without the need for acetic acid. MS signal can then be obtained for each eluting oligonucleotide wherein the MW corresponds to oligo length. Advantageously, this will enable a user to obtain information about each poly(A) species even if the peaks are not completely chromatographically resolved. MS signal can be then obtained for each eluting oligonucleotide, the MW corresponds to oligo length. This will enable to obtain information about each poly(A) species even if the peaks are not completely chromatographically resolved.

In some embodiments, LC MS compatible mobile phases may be optimized to improve resolution power. For example, hexylammonium (HAA), octylammonium (OAA), or tributylammonium (TBA) ion pairing reagents may be used in the mobile phase.

The number of chromatography methods are not limited and may be combined or individually applied based on the method steps discussed above.

### EXAMPLES

### Example 1: IP RP LC MS for RNase T1 Digest

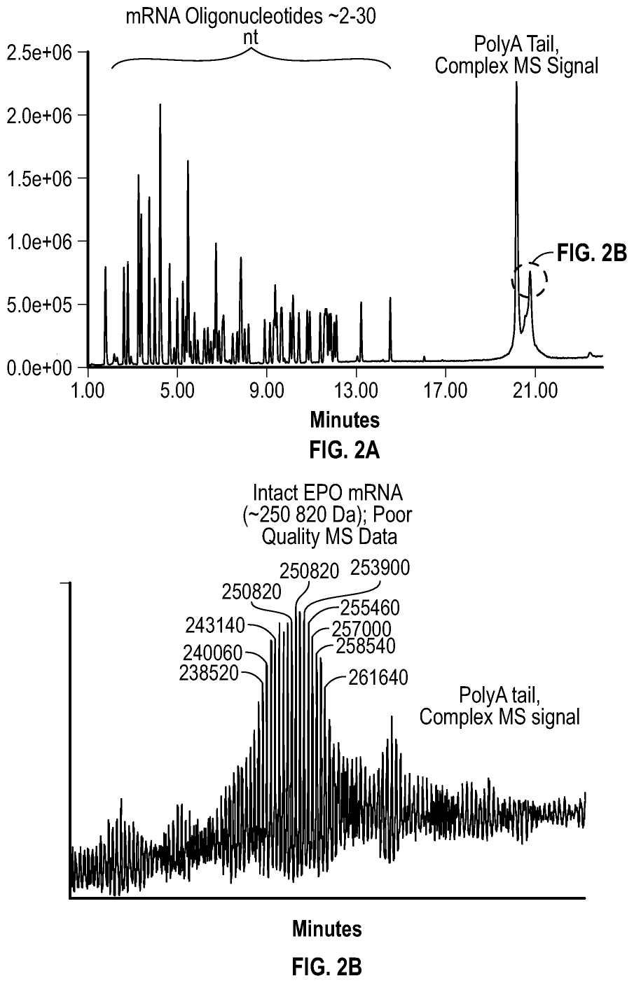

As shown in FIG. 2A, IP RP LC MS analysis was conducted for a RNase T1 digest. Short oligonucleotides eluted between 1-15 minutes. As shown in FIG. 2B, intact mRNA (or dsRNA) appeared at about 20.5 minutes. Poly(A) tail clip elutes as a wide peak near 20.0 min. Its MS data is difficult to interpret due to sample heterogeneity. A number of poly(A) oligonucleotides presumably elute under the peak with maximum at 20.0 min.

The MS conditions used BioAccord MS, Acquity Premier, 2.1×100 mm OST Premier column, 60 C, Flow rate: 300 μL/min, Solvent A: 40 mM HFIP, 8 mM DIPEA (diisopropyl-ethylamine) in DI water, Solvent B: 4 mM HFIP, 4 mM DIPEA in acetonitrile, 0-25% B in 30-min gradient, TUV 260 nm, MS neg ESI mode.

### Example 2: SEC Separation of Poly DNA Synthetic Samples

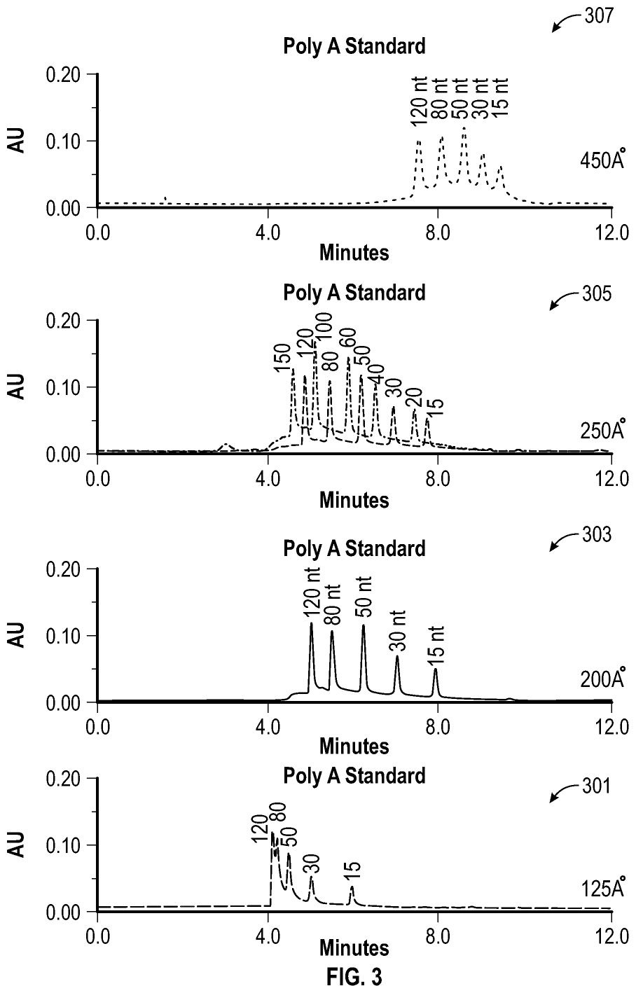

As shown in FIG. 3, SEC pore size optimization results were demonstrated for a 15-150 nt poly DNA standard of synthetic oligonucleotides (crude, not purified).

Conditions for the experiment: The following SEC columns were used—BEH SEC 4.6×150 mm columns commercially available from Waters Technology Corporation having 1.7 μm or 2.5 μm stationary phase (450 Å column), mobile phase: 100 mM phosphate, pH 8, 25° C., 0.2 mL/min, 1 μL injection, TUV 260 nm, for pore size of the sorbent see the right panel.

The sample is Poly DNA standard, 15-150 nt synthetic oligonucleotides (crude, not purified).

Columns:

| | |
|---|---|
| 1. | Protein SEC 125Å, 150 × 4.6 mm, 1.7 μm, 186006505 |
| 2. | Protein SEC 200Å, 150 × 4.6 mm, 1.7 μm, 186005225 |
| 3. | Premier Protein SEC 250Å, 4.6 × 150 mm, 1.7 μm, 186009963 |
| 4. | Protein SEC 450Å, 150 × 4.6 mm, 2.5 μm, 186006851 |

LC conditions: H-class PLUS, 15 μL FTN needle, Premier modified; 100 mM sodium phosphate, pH 8 (6.722 g Na₂HPO₃, 0.318 g NaH₂PO₃, add 500 g water, filter), 0.2 mL/min, 25° C., 260 nm; columns stored in 30% MeOH between experiments.
Sample 1: 15, 30, 50, 80 and 120 mer oligo(A)denosines, 20 pmol/μL in water
Sample 2: 20, 40, 60, 100, 150 mer oligo(A)denosines, 20 pmol/μL in water
Other oligos: 15+30 mer oligoC, 15+30 mer oligo(T), 15-100 mer oligo(T)

Results: the optimal column for poly(A) peaks resolution of around 100 mer is a 250 Å pore size column. Its calibration range for 50-150 mer is highly linear. Other columns are less ideal (125 Å is best suited for relatively short oligos 2-50 mer) and 450 Å column has linear range for 100-1000 mer oligos). 200 Å is suitable for the poly(A) tail analysis in the range 50-150 mer length.

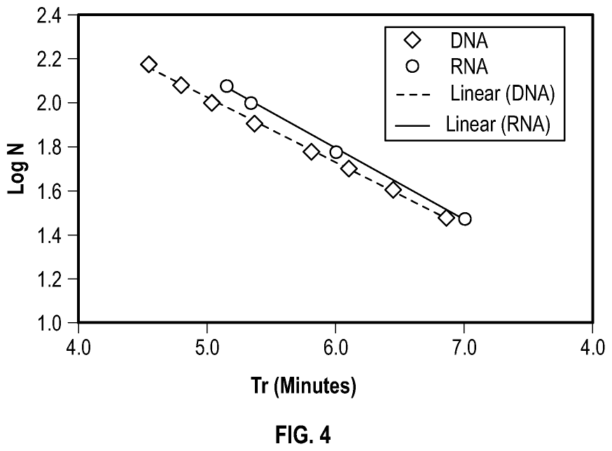

The best separation of a target of 80-120 nt poly(A) range oligonucleotides. FIG. 4 shows an oligo N-mer linear range calibration of a 250 Å SEC column. The linear calibration curves based on DNA or RNA poly(A) standards shown in the FIG. 4 can be used for estimation of observed peak oligo(A) DNA length from its observed retention time tr. Oligo(A) RNA calibration may be different from DNA calibration. Oligo(A) RNA calibration may be applied to measure mRNA poly(A) tail length because the target sample is RNA.

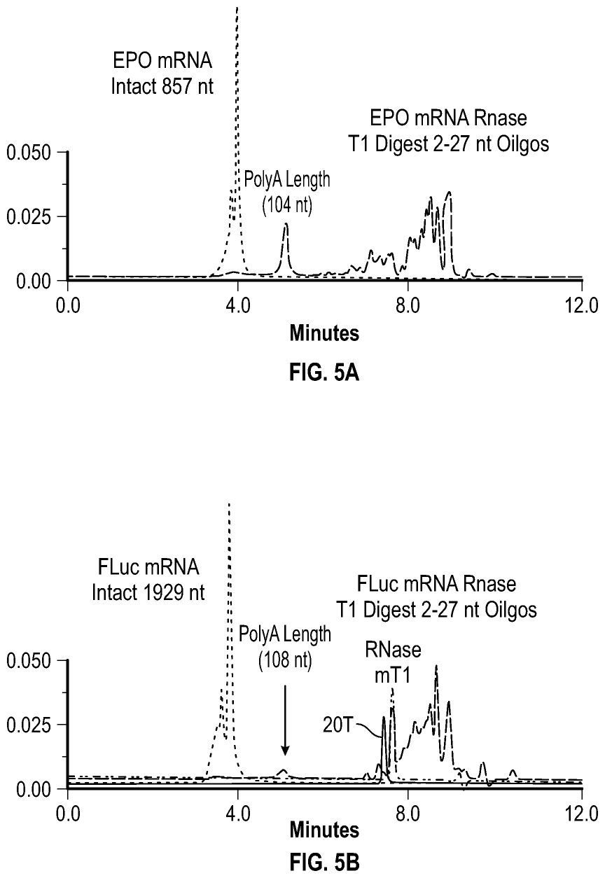

As shown in FIG. 5A, the length of poly(A) tail from EPO mRNA (Trilink L-7209 product) and Fluc mRNA samples were calculated with the above equation. EPO mRNA intact of 857 nt at about 4 minutes was the original mRNA sample prior to sample digestion with T1 RNase enzyme. The poly(A) peak eluting at 5.019 min. The average length N is calculated from the calibrations listed in FIG. 4 as log N=−0.2955×5.019+3.5009=2.0178. 104.2. 409 N is about 10\hat{ }2.0178=104 nt long. The DNA calibration must be adjusted for correct RNA polyA tail length assignment as described later. When RNA calibration shown in FIG. 4 is used, the correct N length of poly(A) species in experiments shown in FIGS. 5A and 5B is about 124 and 127-128 respectively.

As shown in FIG. 5B, RNase T1 sample was added to mRNA and eluted close to 7.7 minutes. An overlay of 20 mer oligo(T) deoxythymidine shows where the short oligos are eluting. The poly(A) released peak elutes at 4.97 min which corresponds to 128 nt length.

SEC analysis was extremely robust. Over several months, minimal change was observed to the separation (calibration) with poly(A) DNA standard sample.

Experiments were conducted to assess RNA poly(A) (100 mer oligo(A) RNA) and compare retention of 100 nt RNA with 100 nt DNA.

Experimental conditions: 100 mM phosphate, pH 8, 25° C., 0.2 mL/min, 1 ul injection, TUV.

DNA oligo(A) standard may be used in lieu of RNA oligo(A) but would require adjustment of the calibration using a conversion such as by adjusting the slope of calibration curve as shown in FIG. 4 and modifying the intercept. SEC 250 Å RNAse calibration standards of DNA/RNA were conducted to account for this variation. Those two adjustments (slope, intercept) can be deduced from the linear relationship between DNA and RNA calibrations.

Based on the data plotted in FIG. 4, DNA to RNA calibration conversion is as follows: Adjust DNA linear slope by 0.2955-0.31161 value. Adjust the DNA calibration intercept by 3.65578-3.5009 value. N mer (nt) value is than calculated from DNA experimental calibration that was adjusted to RNA equation. N length is calculated as N=10\hat{ }(Log N). Log N=−0.2955×tr+3.5009 (DNA calibration) and Log N=−0.31161×tr+3.65578 (RNA calibration).

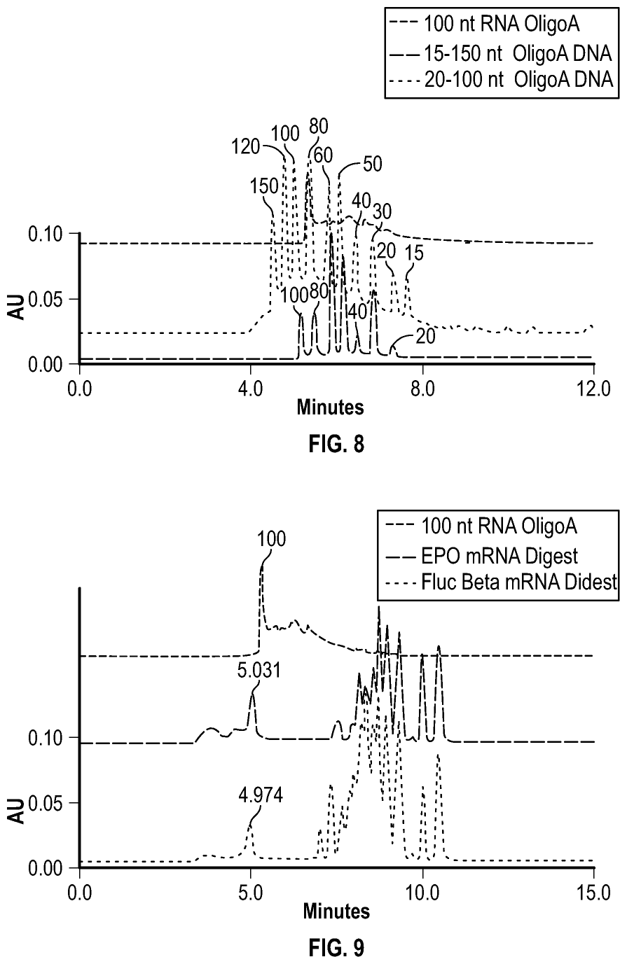

As shown in FIG. 8, while different sequence DNA length standards (oligo(T), oligo (A)) give similar calibration, RNA 100 nt oligo(A) is shifted. Results of RNA poly(A) length is underestimated when using oligo(A) DNA calibration without correction. This means that oligo(A) DNA is not a surrogate for RNA oligo(A) calibration without correction (two-point or multi-point conversion).

As shown in FIG. 9, EPO and Flue beta were tested with 100 nt RNA oligo(A). Previously assigned poly(A) length based on oligo(A) DNA calibration was about 104 nt. With oligo(A) RNA standard calibration, the poly(A) peak is >120 nt.

The reason DNA to RNA calibration conversion may be useful is because DNA oligo(A) standards are easy to synthesize, inexpensive, and are higher purity compared with RNA and stable in solution. Therefore, it makes sense to use DNA calibration standard and adjust it for RNA sample analysis, if good RNA standard is not readily available.

### Example 3: LC MS Analysis Example of EPO and Fluc Beta Poly(A) Tail

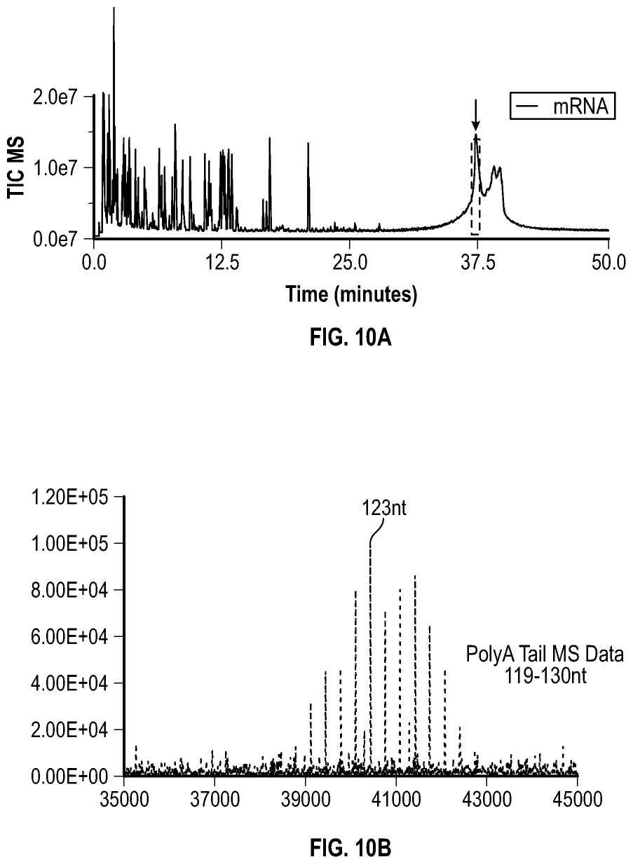

As shown in FIG. 10A, LC MS was used to analyze poly(A) tail (EPO mRNA T1 RNase digest). While the chromatogram shows that the poly(A) RNA species all coelute in a broad peak, the MS can reveal that under that wide peak is multiple poly(A) species with different length and intensity. LC MS with deconvolution is a qualitative analysis rather than quantitative, requiring a skilled analyst. The intensity of the deconvoluted masses depends on the parameters of the deconvolution software. In this case, as shown in FIG. 10B, the poly(A) tail length was 119-130 nt.

As shown in FIG. 11A, LC MS was also used to analyze the poly(A) tail of Flue beta mRNA T1 RNase digest. In this case, as shown in FIG. 11B, the poly(A) tail length was 120-128 nt.

Conditions: ACQUITY Premier Peptide BEH C18, 2.1×150 mm, 300 Å, 1.7 μm, H-Class Premier system, Vion MS, negative mode, cone 40 V, 400-5000 m/z, de-solvation temp 400 C.

Solvent A: 1% HFIP, 0.1% DIPEA in DI water, Solvent B: 0.075% HFIP, 0.0375% DIPEA in acetonitrile.

Gradient 3 to 30% B in 60 min, 60.5 min 95% B wash, 0.4 mL/min, 70° C.

Results: the peak highlighted with red arrow is a poly(A) tail. Large front contains multiple shorter poly(A) species that may not be present in sufficient intensity to be detected by MS.

When summing only peak top, partial poly(A) species envelope is obtained.

Deconvolution is qualitative. Relative peak height varies due to deconvolution algorithm and deconvolution parameters.

### Example 4: Octylammonium Acetate IP System

FIG. 12A shows results from a LC UV analysis of liberated poly(A) tail using a high resolution octylammonium acetate IP system. Poly(A) species in the range of ˜100-140 can be resolved and their length assigned using a size standard oligo(A) RNA, e.g. 100 nt and its shorter RNA oligo(A) species.

Conditions: 2.1×150 mm, 1.7 um BEH C18 300 Å Peptide Premier column. The 86-101 nt peaks (100 nt synthetic oligo(A) RNA standard) were used for calibration. The length of poly(A) tail was assigned using this calibration. Polydispersity of poly(A) tail liberated from mRNA is clearly visible. As shown in FIG. 12A, Flue Beta mRNA has a longer poly(A) tail than EPO mRNA sample. A calibration standard can be used to generate a calibration curve and to calculate poly(A) length N and distribution in the poly(A) tail analysis such as shown in FIG. 12B.

### Example 5: SEC Calibrations Obtained for RNA and DNA Oligonucleotides

SEC calibration was used to compare more expensive RNA oligo(A) versus more available DNA oligo(A) standards. In this example, linearity of 250 Å SEC columns with a DNA oligo(A) standard having a mixture of 5 to 150 nt oligodeoxyribonucleotide adenosine mixtures was evaluated. For simplicity, the calibration curve was directly plotted as a function of retention time versus log N (N is the number of nucleotides).

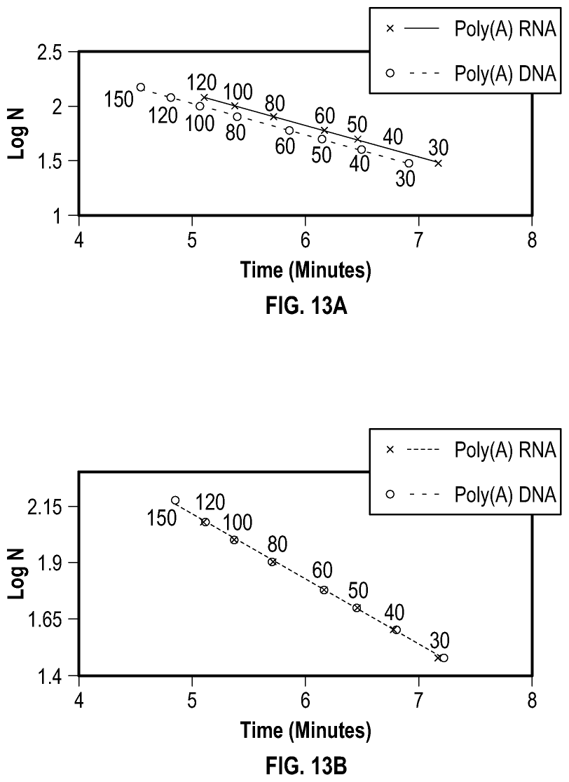

As shown in FIG. 13A, the calibration curve was linear for the DNA oligo(A) standard with a coefficient of determination R2˜0.999. The calibration plots in FIG. 13A include 30-150 nt data points. Shorter standards are visibly deviating from the linearity. This means that the length of the DNA oligo(A) standard oligonucleotides can be accurately measured from their SEC retention time. The choice of DNA oligo(A) oligonucleotides for SEC calibration was motivated by their easy availability. Synthetic DNA oligo(A) oligonucleotides are relatively inexpensive and can be purchased from vendors in high quality up to 150 nt in length. Because of the retention shift of different types of oligonucleotides, RNA oligo(A) standards are beneficial because they are chemically consistent with the poly(A) RNA tail. The prepared oligoribonucleotide(A) calibration as shown in FIG. 13A can be directly used for poly(A) RNA tail length SEC measurement.

To circumvent the problem to use expensive RNA oligo(A) standards as calibrants for poly(A) tail RNA measurement, two linear calibrations were constructed for the same column using DNA oligo(A) and RNA oligo(A) standards. As can be seen in FIG. 13A, the calibrations have essentially the same slope with a small difference in the intercept. This means that a retention time correction factor can be applied to superimpose the DNA oligo(A) calibration onto the RNA oligo(A) calibration curve. The concept of using DNA oligo(A) standards as surrogates for RNA oligo(A) calibrants in SEC was validated with two UPLC systems and three SEC columns. The experiments confirmed that the DNA oligo(A) and RNA oligo(A) calibrations are parallel.

Table 1 lists the retention data of four SEC experiments with DNA oligo(A) and RNA oligo(A) calibrants. The average retention difference between RNA oligo(A) and DNA oligo(A) represents the proposed correction factor Δtᵣ of 0.306 min. While the tubing volume and injection loop size of LC instrument affect the absolute retention times of calibrants in SEC, the relative retention difference between RNA oligo(A) and DNA oligo(A) standards remains constant.

TABLE 1
SEC CALIBRATIONS ΔT_{R} CORRECTION FACTOR ESTIMATION

| | UPLC 2 | UPLC 1 | UPLC 1 | UPLC 1 | UPLC 2 | UPLC 1 | UPLC 1 | UPLC 1 | UPLC | UPLC | UPLC | UPLC |
|---|---|---|---|---|---|---|---|---|---|---|---|---|
| | DNA | DNA | DNA | DNA | RNA | RNA | RNA | RNA | 2 | 1 | 1 | 1 |
| | oligo(A) | oligo(A) | oligo(A) | oligo(A) | oligo(A) | oligo(A) | oligo(A) | oligo(A) | Δtᵣ | Δtᵣ | Δtᵣ | Δtᵣ |
| Length | col 1 | col 1 | col 2 | col 3 | col 1 | col 1 | col 2 | col 3 | col 1 | col 1 | col 2 | col 3 |
| (nt) | tᵣ (min) | tᵣ (min) | tᵣ (min) | tᵣ (min) | tᵣ (min) | tᵣ (min) | tᵣ (min) | tᵣ (min) | (min) | (min) | (min) | (min) |
| 150 | 4.535 | 4.514 | 4.55 | 4.543 | | | | | | | | |
| 120 | 4.801 | 4.781 | 4.824 | 4.81 | 5.093 | 5.068 | 5.117 | 5.104 | 0.292 | 0.287 | 0.293 | 0.294 |
| 100 | 5.047 | 5.028 | 5.078 | 5.065 | 5.359 | 5.336 | 5.389 | 5.374 | 0.312 | 0.308 | 0.311 | 0.309 |
| 80 | 5.376 | 5.358 | 5.416 | 5.397 | 5.698 | 5.672 | 5.736 | 5.717 | 0.322 | 0.314 | 0.32 | 0.32 |
| 60 | 5.822 | 5.806 | 5.875 | 5.856 | 6.139 | 6.115 | 6.189 | 6.165 | 0.317 | 0.309 | 0.314 | 0.309 |
| 50 | 6.107 | 6.092 | 6.168 | 6.144 | 6.43 | 6.407 | 6.485 | 6.46 | 0.323 | 0.315 | 0.317 | 0.316 |
| 40 | 6.448 | 6.434 | 6.519 | 6.495 | 6.747 | 6.714 | 6.802 | 6.775 | 0.299 | 0.28 | 0.283 | 0.28 |

Retention of DNA oligo(A) and RNA oligo(A) standards was measured with three SEC columns and two UPLC systems (UPLC 1 and UPLC 2). DNA oligo(A) standards retention times are shown in the left table section and RNA oligo(A) retention times in the center table section. Calculated retention time difference Δtᵣ between RNA oligo(A) and DNA oligo(A) is in the right table section. The retention time correction factor was calculated as Δtᵣ=tᵣ (RNA oligo(A))−tᵣ (DNA oligo(A)). Δtᵣ retention factor was calculated by averaging the Δtᵣ data form all four experiments; its average value is 0.306 min.

Experimental conditions: ACQUITY Premier Protein SEC Column, 250 Å, 1.7 μm, 4.6×150 mm. Flow rate 0.2 mL/min, column temperature 25° C., detection UV 260 nm. UPLC system 1 was H-class, QSM (Quaternary Solvent Manager), FTN (Flow Through Needle) Sample Manager was equipped with 15 mL needle. UPLC system 2 was H-class with BSM (Binary Solvent Manager), and FL (Fixed Loop) Sample Manager equipped with 5 mL loop. Difference in LC instrument tubing length, i.d., and sample loop volume can offset the absolute retention times of oligo(A) retention standards. The correction factor is a relative value and remains constant even when different LC systems are used for the SEC experiment.

As shown in FIG. 13B, when the correction factor Δtᵣ=0.306 min is used to adjust experimental DNA oligo(A) retention times, both DNA oligo(A) and RNA oligo(A) SEC calibration curves become virtually identical. Therefore, it is possible to utilize inexpensive, readily available, and chemically stable DNA oligo(A) oligonucleotides as surrogates for the RNA oligo(A) calibration standards. In the simplest implementation, the analyst can add the Δtᵣ=0.306 min correction factor to the measured DNA oligo(A) retention times prior to plotting the calibration curve log N=a×tᵣ+b. The resulting SEC calibration is directly applicable for mRNA poly(A) tail measurement.

What is claimed is:

1. A method of determining a mRNA poly(A) tail size comprising:
digesting an mRNA molecule to liberate a poly(A) tail;
preparing a chromatographic sample comprising the mRNA poly(A) tails;
preparing a second chromatographic sample comprising a reference sequence comprising a mRNA poly(A) tails having a predetermined length;
separating the first and second samples by a chromatography method, which result in one or more chromatograms; and
determining a sequence length of the mRNA poly(A) tails by comparing the chromatograms of the first and second samples.

2. The method of claim 1, wherein the reference sequence comprises 30-150 nt in length.

3. The method of claim 1, further comprising selecting a length of the reference sequence by calibrating with a DNA or RNA reference sequence standard.

4. The method of claim 1, further comprising preparing the poly(A) tail reference sequence standard by mixing and digesting poly(A) synthetic oligonucleotides and digesting the poly(A) synthetic oligonucleotides or by extending the reference sequence using poly(A)denylase enzymes to create a full ladder of poly(A) species.

5. The method of claim 1, further comprising using the poly(A) tail reference sequence standard to generate a calibration curve and determining the length of a poly(A) clip from the mRNA molecule.

6. The method of claim 1, wherein the mRNA poly(A) tails range between about 80 to about 120 oligonucleotides.

7. The method of claim 1, wherein the mRNA poly(A) tails have a length distribution that is observable from chromatogram with a n/n−1 resolution.

8. The method of claim 1, wherein the method further comprises calculating the dispersity of the mRNA poly(A) tails based on one or more poly(A) tail peak widths of the chromatograms.

9. The method of claim 1, wherein the chromatography method is performed with mass spectrometry (MS) compatible mobile phases.

10. The method of claim 1, wherein the digesting step comprises liberating the 3′ poly(A) tail of the mRNA molecule by enzymatic or chemical cleavage.

11. The method of claim 1, wherein the mRNA poly(A) reference sequence length is about 100 nucleotides.

12. The method of claim 1, wherein the chromatography method is selected from the group consisting of ultraviolet size-exclusion chromatography (SEC UV), ultraviolet ion-pair reversed-phase liquid chromatography (IP RP LC UV), ultra high performance liquid chromatography (UPHLC), and a combination thereof.

13. The method of claim 12, wherein the chromatography method is UPHLC.

14. The method of claim 12, wherein the chromatography method is SEC UV.

15. The method of claim 12, wherein the chromatography method is IP RP LC UV.

16. The method of claim 12, wherein the chromatography method is a combination of UPHLC, SEC UV, and IP RP LC UV.

17. The method of claim 13, wherein the SEC UV and IP RP LC UV is performed after UPHLC.
